(19) United States
(12) Patent Application Publication
(10) Pub. No.: US 2019/0392940 A1
(43) Pub. Date: Dec. 26, 2019

(54) MEDICAL IMAGE DISPLAY SYSTEM

(71) Applicant: KONICA MINOLTA, INC., Tokyo (JP)

(72) Inventors: Kenichirou SUZUKI, Tokyo (JP); Akira KURAHASHI, Tokyo (JP)

(21) Appl. No.: 16/447,031

(22) Filed: Jun. 20, 2019

(30) Foreign Application Priority Data
Jun. 22, 2018 (JP) 2018-118372

(51) Int. Cl. G16H 30/00; A61B 6/03; A61B 6/00

(52) U.S. Cl. CPC G16H 30/00; A61B 6/032; A61B 5/7425; G06T 2210/41; A61B 6/463; A61B 6/464

(57) ABSTRACT

A medical image display system includes a client and a server. The client is able to display a plurality of images on a display. The server includes a hardware processor which stores data of a plurality of images in a storage, which obtains environment information regarding a viewing environment of the image, which determines an image transfer condition regarding transfer of the image to the client based on the obtained environment information, which selects as target images at least some of the plurality of images stored in the storage based on the image displayed on the client and the determined image transfer condition, and which transfers the selected target images to the client using a communicator.

### CROSS REFERENCE TO RELATED APPLICATIONS

The present invention claims priority under 35 U.S.C. § 119 to Japanese Application No. 2018-118372 filed Jun. 22, 2018, the entire content of which is incorporated herein by reference.

### BACKGROUND

### 1. Technological Field

The present invention relates to a medical image display system.

### 2. Description of the Related Art

Usually, medical images such as tomographic images imaged by using computer tomography (CT) or magnetic resonance imaging (MRI) and dynamic images imaged by using a radiation image imaging apparatus such as a flat panel detector (FPD) are stored and managed on a server, and these can be viewed as necessary when the image is downloaded and viewed on a client.

By using a small terminal such as a tablet as a client and having a medical doctor always carrying this client, the medical doctor can read a medical image on the spot when the medical doctor is requested outside the medical facility to diagnose an image in an emergency.

When a patient is transported in an emergency in an unconscious state and the patient is imaged, the whole body of the patient may be imaged by a tomographic image just in case. When a specific site of the lungs, etc. is imaged a plurality of times, or the medical doctor desires to observe the dynamic state of the lungs, the dynamic images may be imaged continuously for a comparatively long period of time. The number of medical images imaged in such situation becomes huge. Therefore, depending on the situation of the network that the client is connected to or depending on how the user views the images, a large amount of time may be needed to download the medical image, and it may be difficult to view the images smoothly. Such problem may especially occur in emergencies where connection may be overloaded. Such problem may also occur in an environment where the medical image is viewed by a cloud.

Japanese Patent No. 6279401 proposes replacing some of the original images with a subtraction image based on parameters such as speed of the network connection or the speed that the user advances the images in order to optimize the size of the transferred images.

Lately, there is a zero footprint technique in which installing an application is not necessary and the medical image can be viewed on a browser without storing the medical image. There is also a server rendering technique in which the image processing with a high load is performed on the server and then the image is delivered to the client. By using such techniques, it is becoming a suitable environment to read medical images safely regardless of time or place even if a terminal with low processing capabilities and low costs is used as the client.

However, the memory region usable by the browser software is small in the client employing such zero footprint technique. Therefore, when a plurality of medical images are successively downloaded, the medical images downloaded in the beginning may be successively destroyed and wasted.

When the server rendering technique is employed, and the image processing of the medical image being viewed is requested to the server while the plurality of downloaded medical images are being viewed, the medical images processed by image processing are downloaded in the same number as the viewed medical images and are switched with the original medical images. Then, the medical images which were already downloaded but which are not yet viewed are wasted.

When the client is used outside the medical facility, the medical images are delivered by communication through mobile phone carriers. However, in communication through mobile phone carriers, when the amount of communication within a certain period of time reaches a predetermined value, the speed of transferring data is drastically restricted. If a huge amount of medical images are delivered under such conditions, the transfer speed may be restricted immediately.

Alternatively, if only the medical images displayed immediately are downloaded one image at a time, unnecessary downloads and the concern for the possibility of the communication being limited can be decreased. However, the speed (for example frame/second: fps) that the displayed images are switched will always be slow, and convenience may decrease.

### SUMMARY

The present invention is made in view of the above problems, and the object of the present invention is to reduce volume of downloading wasted medical images which are not viewed without reducing speed of switching displayed images in a medical image display system provided with a client which is able to download a plurality of medical images and which is able to sequentially display the plurality of medical images.

To achieve at least one of the abovementioned objects, according to an aspect of the present invention, a medical image display system reflecting one aspect of the present invention is described, the medical image display system comprising a client which is able to display a plurality of images on a display; and a server including a hardware processor which stores data of a plurality of images in a storage, which obtains environment information regarding a viewing environment of the image, which determines an image transfer condition regarding transfer of the image to the client based on the obtained environment information, which selects as target images at least some of the plurality of images stored in the storage based on the image displayed on the client and the determined image transfer condition, and which transfers the selected target images to the client using a communicator.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention.

FIG. 1 is a block diagram showing a schematic configuration of a medical image display system according to an embodiment of the present invention.

FIG. 2 is a block diagram showing a specific configuration of a server included in the medical image display system shown in FIG. 1.

FIG. 3 is a block diagram showing a specific configuration of a client included in the medical image display system shown in FIG. 1.

FIG. 4 is an example of image transfer condition setting data stored in the storage of the server shown in FIG. 2.

FIG. 5 is a flowchart showing a flow of an initial image request process performed by the client shown in FIG. 3.

FIG. 6 is a flowchart showing a flow of a display-in-progress image request process performed by the client shown in FIG. 3.

FIG. 7A, FIG. 7B, and FIG. 7C are diagrams describing a relation between an image displayed on the client and a transferred target image.

### DETAILED DESCRIPTION OF EMBODIMENTS

Hereinafter, one or more embodiments of the present invention will be described with reference to the drawings. However, the scope of the invention is not limited to the disclosed embodiments.

[Image Display System]

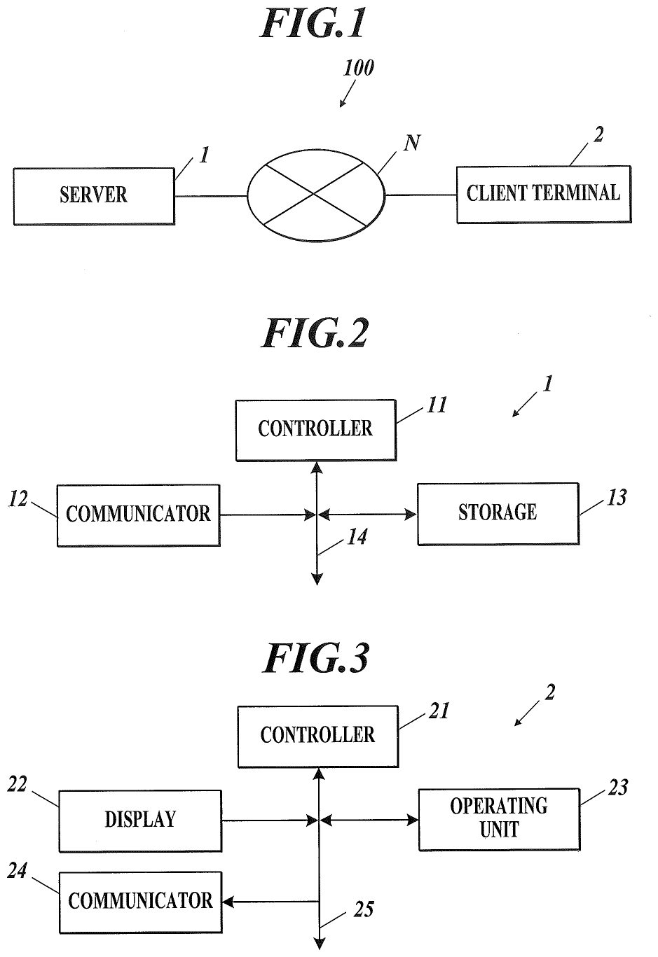

First, a configuration of a medical image display system (hereinafter, system 100) according to the present embodiment is described. FIG. 1 is a block diagram showing a schematic configuration of the system 100.

For example, as shown in FIG. 1, the system 100 according to the present embodiment includes a server 1 and a client 2.

The server 1 and the client 2 are connected to each other through a communication network N.

The server 1 is connected through a modality not shown (for example, computed tomography (CT), magnetic resonance imaging (MRI), and flat panel detector (FPD), etc.) directly or through a communication network N. The server 1 is able to obtain medical images of a subject generated in the modality in the form of data from the modality.

The server 1 may be a configuration used independently or may be a part of another system (for example, a medical image management system (picture archiving and communication system, PACS)).

The details of the server 1 is described later.

A user (for example, medical doctor) views the medical image using the client 2.

The client 2 is able to communicate with the server 1 through the communication network N.

The client 2 according to the present embodiment is able to perform communication through mobile phone carriers to communicate data, etc. with the server 1 using a mobile phone communication line. Therefore, the use of the client 2 is not limited to inside a medical facility, and can be used anyplace where a mobile phone can be used including a home of a homecare patient.

FIG. 1 is an example showing the system 100 with only one client 2 connected to the server 1. Alternatively, a plurality of clients 2 can be connected to the server 1.

According to the present embodiment, the client 2 is able to use the mobile phone communication line but alternatively, the client 2 may be a device which does not include the function to use the mobile phone communication line.

Details of the client 2 is described later.

[Server]

Next, the server 1 is described in detail. FIG. 2 is a block diagram showing a specific configuration of the server 1.

For example, as shown in FIG. 2, the server 1 includes a controller 11, a communicator 12, and a storage 13, and the controller 11, the communicator 12, and the storage 13 are connected to each other through a bus 14.

The server 1 may be a cloud server provided in a cloud environment.

Alternatively, the server 1 may be provided with a display or operating unit which are not shown. With this, the medical image may be displayed on the server 1 or various image processes may be displayed in the server 1.

Alternatively, a display or operating unit which are not shown may be provided near the server 1. With this, an image processing apparatus which is able to display the medical image or perform various image processes may be configured near the server 1.

The controller 11 includes a CPU (central processing unit), a RAM, or the like, and centrally controls the processing operation of each unit of the server 1. Specifically, the CPU reads various processing programs stored in the storage 13 and deploys the program in the RAM according to various signals from the client 2 received through the communicator 12. With this, various processes are performed in coordination with the programs.

The communicator 12 includes a network interface, and transmits and receives data with external devices (client 2, etc.) connected through a communication network N such as a LAN (local area network), WAN (wide area network), the internet, etc.

The communicator 12 according to the present embodiment includes an antenna (not shown) to transmit and receive the radio wave with mobile base stations.

The storage 13 includes a HDD (Hard Disk Drive) or a semiconductor nonvolatile memory, etc., and stores various processing programs and parameters and files necessary to execute such programs.

According to the present embodiment, the following programs are stored, for example, a web server program which realizes the function as a web server in which the web server provides various web screens to the web browser and a web application program which operates on the web server and which provides the medical images to the user of the client 2 through the web browser.

The storage 13 according to the present embodiment stores image transfer condition setting data in the image transfer condition data region.

The image transfer condition setting data according to the present embodiment is a table showing a relation of correspondence between the plurality of environment information and the plurality of image transfer conditions.

The “environment information” includes at least any of the following, user name, client information (for example, ID number, etc.), communication speed between the server 1 and the client 2, connection state between the server 1 and client 2 (for example, wired communication or wireless communication, carrier connection or Wi-Fi connection, etc.), speed of switching the image on the display 22, department where the user belongs (emergency or not, etc.), modality used in imaging the image, number of images included in one file, color of image (color or monochrome, etc.).

Other than the above, the information regarding the device which is the source of transmission or the destination of transmission can be referred. For example, the process to display the image after transferring the image changes depending on the specifications (CPU, image resolution, memory) for the type of device which is the destination of transmission.

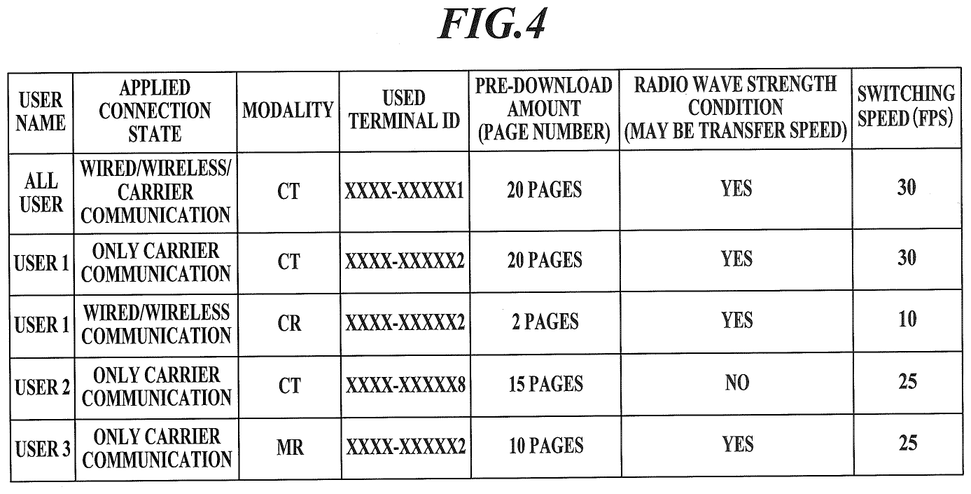

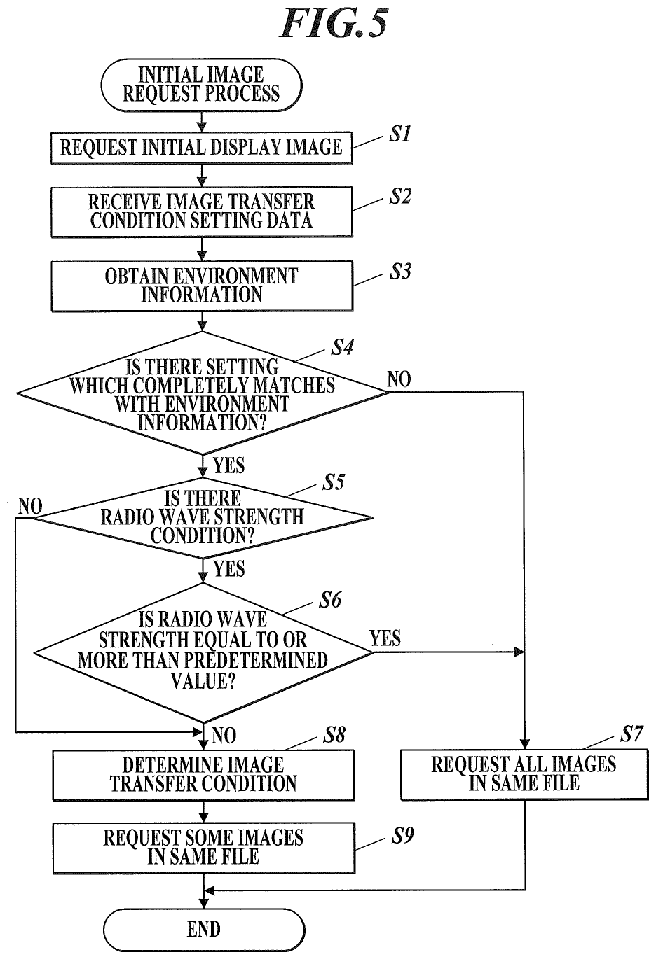

The image transfer condition setting data according to the present embodiment includes the following as the environment information as shown in FIG. 4, the user name, connection state, modality used in imaging, information of client 2, radio wave strength condition, and switching speed of image. The environment information included in the table can be suitably changed.

The “image transfer condition” according to the present embodiment is the number of pre-downloads of the image. The amount of data or format of compression of the image can be included in the image transfer condition.

Here, “pre-download” means to download the medical image which is displayed by switching after the medical image displayed at present on the client 2 or the medical image which is to be immediately displayed.

The storage 13 is able to store the image file of the medical image in the image data region.

Here, “image file” means a group of a plurality of medical images obtained in one occasion of imaging. That is, the storage 13 may store the data of a plurality of images included in one file, or a group of a plurality of medical images associated to each other.

Here, “plurality of medical images” include dynamic images, a plurality of tomographic images, or other plurality of radiation images.

The “dynamic images” are images including a plurality of frames obtained by imaging a predetermined site of the subject repeatedly.

The “plurality of tomographic images” are obtained by imaging cross-sections in which a plurality of planes cut a subject. The plurality of planes are positioned orthogonal to a predetermined direction and are aligned in the predetermined direction with a predetermined interval in between.

The “radiation images” are images obtained by imaging the subject a plurality of times.

A combination of the above images can be transmitted.

The storage 13 according to the present embodiment stores data of a plurality of related images relating to the plurality of images in the image data region.

For example, the “related images” are medical images after processing the above images with a predetermined image process, medical images of the same subject obtained by performing imaging similar to the above with different imaging conditions (for example, using contrast mediums), or medical images of the same subject obtained by performing imaging similar to the above on a different day.

The storage 13 according to the present embodiment stores screen layout information in the image data region.

The “screen layout information” includes information regarding the number of regions that the original display region is divided into and the relation of correspondence between the display region and the display image.

Here, the image transfer condition setting data region and the image data region are provided in one storage 13 but alternatively, two individual storages can be provided to store each of the image transfer condition setting data and the image data.

The server 1 configured as described above includes the following functions performed by the controller 11 executing software processes in coordination with the programs stored in the storage 13.

For example, the controller 11 includes the function to transmit to the client 2 through the communicator 12 the image transfer condition setting data stored in the storage 13 when an image request signal requesting a later-described initial display image is received from the client 2.

The controller 11 includes a function to select at least some of the plurality of images stored in the storage 13 as target images based on the image displayed on the display 22 of the client 2 and the image transfer condition determined by the client 2.

When the plurality of images is the dynamic image, the target images are a predetermined number of frames not displayed yet obtained by imaging a plurality of times in a time period in at least either one of directly before or directly after a frame displayed on the display 22 of the client 2.

When the plurality of images are a plurality of tomographic images, the target images are a predetermined number of tomographic images not displayed yet obtained by imaging a predetermined number of cross-sections aligned in a direction in at least either one of a predetermined direction or opposite of the predetermined direction from the tomographic image displayed on the display 22 of the client 2.

According to the present embodiment, when the above-described related images are stored in the storage 13, at least some of the plurality of images are selected as the target images and the related images corresponding to the target images (for example, same serial number or the imaged phase of the operation is the same) are selected as the target related images.

The controller 11 according to the present embodiment includes the function to render the data of the selected target images or the target related images to generate delivered images. This is known as the server rendering function.

When the terminal with a high processing ability is used as the client 2, the rendering is performed in the client 2 to generate the displayed images.

The controller 11 includes the function to transfer the selected target images and the target related images to the client 2 through the communicator 12.

According to the present embodiment, the server 1 performs the rendering, and therefore the generated delivered images are transferred to the client 2.

According to the present embodiment, the image layout information is transferred together with the delivered images.

The controller 11 according to the present embodiment includes a function to compress the images by lossless format or lossy format based on the image request signal from the client 2.

Here, the “lossless format” is a format of compression in which the data after compressing and deploying ZIP, JPEG2000, etc. for example, is a complete match to the original data.

The “lossy format” is a format of compression which converts raw data obtained by imaging with a modality to a format with a small data amount in order to transmit and view the data quickly.

The controller 11 according to the present embodiment includes the function to transfer all of the images included in a file to the client when the predetermined conditions are satisfied.

The “predetermined conditions” are described later.

[Client]

Next, the details of the client 2 are described. FIG. 3 is a block diagram showing the specific configuration of the client 2.

As shown in FIG. 3, the client 2 includes a controller 21, a display 22, an operating unit 23, and a communicator 24. The controller 21, the display 22, the operating unit 23, and the communicator 24 are connected to each other through a bus 25.

Preferably, the client 2 is a device which is portable or wearable by the user, for example, a mobile phone, a tablet terminal, a note-type PC, a head-mount display or the like.

The client 2 according to the present embodiment is a terminal in which an application is not installed and a storage with a large capacity is not provided. No data (footprints) remains in the application delivered from the server 1. That is, the client 2 according to the present embodiment is a zero footprint terminal.

The controller 21 according to the present embodiment includes a CPU for zero footprint or a memory with a small capacity. The controller 21 centrally controls the processes in each unit of the client 2. Specifically, the controller 21 reads the web application programs stored in the storage 13 of the server 1 and executes the program to transmit various display signals to the display 22 according to the operation signals input from the operating unit 23.

The display 22 includes a monitor such as a LCD and displays various screens according to the instruction of the display signal input from the controller 21.

That is, based on the display data of various web screens received from the server 1, the display 22 is able to display the list of examinations, and the medical image corresponding to the selected examination, for example.

The operating unit 23 includes a pointing device such as a keyboard or a mouse including various keys or a touch panel layered on the display 22, and operation signals input according to a key operation on the keyboard or the mouse operation or the position touched by the touch operation on the touch panel are output to the controller 21.

The communicator 24 includes a network interface and transmits and receives data with external devices (server 1) connected through the communication network N, such as the LAN, WAN, and the internet.

The communicator 24 according to the present embodiment includes an antenna which is not shown to transmit and receive radio waves with a mobile base station.

The controller 21 of the client 2 displays a list screen which is a list of examinations performed until now on the display 22 when the power is turned ON, for example.

The controller 21 switches the display of the display 22 from the list screen to a viewer screen to view the images when the predetermined screen switching operation is performed in a state with the list screen displayed.

The controller 21 switches the display of the display 22 from the viewer screen to the list screen when a predetermined return operation is performed in the state with the viewer screen displayed.

(1. Operation to Switch from List Screen to Viewer Screen)

The controller 21 performs the initial image request process when the above-described predetermined screen switching operation is performed.

In the initial image request process, as shown in FIG. 5, first the image request signal to request the initial display image which is the medical image displayed first on the viewer screen is transmitted to the server 1 through the communicator 24 (step S1).

In step S1 according to the present embodiment, the information request signal to request screen layout information is transmitted with the image request signal.

After requesting the initial display image, the image transfer condition setting data is received from the server 1 through the communicator 24 (step S2).

The received display image data is temporarily stored in the memory.

After the image transfer condition setting data is received, the environment information is obtained (step S3).

Here, the environment information provided in the image transfer condition setting data is obtained by measuring or analyzing the signals.

After the environment information is obtained, the image transfer condition setting data is referred, and it is determined whether there is a setting which matches with all of the obtained environment information in the plurality of settings (combination of environment information (here, connection state, client information, and modality)) described in the data (step S4).

Here, the referred environment information is to be three types such as connection state, client information and modality, but this may be two types or less or four types or more.

Here, it is determined whether all of the environment information match, but it is possible to determine whether the number of items matches or a percentage matches at a predetermined value or more.

In step S4, learning is performed each time the environment information is obtained, and the result can be reflected on the image transfer condition setting data.

In step S3, when it is determined that among the plurality of settings described in the image transfer condition setting data, there is a setting which matches with all of the obtained environment information (step S4), Yes), it is determined whether there is a radio wave strength condition in the environment information (step S5).

In step S5, when it is determined that there is no radio wave strength condition (step S5; No), the process jumps to step S8.

In step S5, when it is determined that there is a radio wave strength condition (step S5; Yes), it is determined whether the radio wave strength is a predetermined value or more (step S6).

In step S6, when it is determined that the radio wave strength is a predetermined value or more (step S6; Yes), or in step S4, when it is determined that among the plurality of settings described in the image transfer condition setting data, there are no settings which match with all of the obtained environment information (step S4; No), the image request signal requesting all of the medical images included in the same file as the displayed initial display image is transmitted to the server 1 through the communicator 24 (step S7), and the initial image request process ends.

The radio wave strength in steps S5 and S6 may be the transfer speed.

In step S6, when it is determined that the radio wave strength is not the predetermined value or more (it is less than the predetermined value) (step S6; No), the image transfer condition regarding the transfer of the image to the client is determined based on the obtained environment information (step S8).

According to the present embodiment, the table is referred and the number of pre-downloads corresponding to the obtained environment information is determined as the image transfer condition.

After the image transfer condition is determined, the image request signal requesting the images included in the same file as the displayed image in an amount defined in the image transfer condition is transmitted to the server 1 through the communicator 24 (step S9) and the initial image request process ends.

The present embodiment describes performing all of the processes in steps S4 to S6 but when only the radio wave strength is obtained as the environment information, there is no need to perform the process in step S4, and when the radio wave strength is not obtained as the environment information, there is no need to perform the processes in steps S5 and S6.

(2. Operation when Display Image is Switched)

When the predetermined image switching operation is performed in the state with the viewer screen (initial display image) displayed, the controller 21 performs a display-in-progress image requesting process.

Here, “image switching operation” is to swipe the screen or to touch a predetermined play icon when the operating unit 23 is a touch panel, or dragging and rotating of the wheel when the operating unit 23 is the mouse. The “image switching operation” is operation to switch the display from the medical image being displayed to another medical image which is not displayed yet (flip through displayed images).

The display-in-progress image requesting process is performed each time the screen switching operation is performed while the viewer screen is displayed on the display 22.

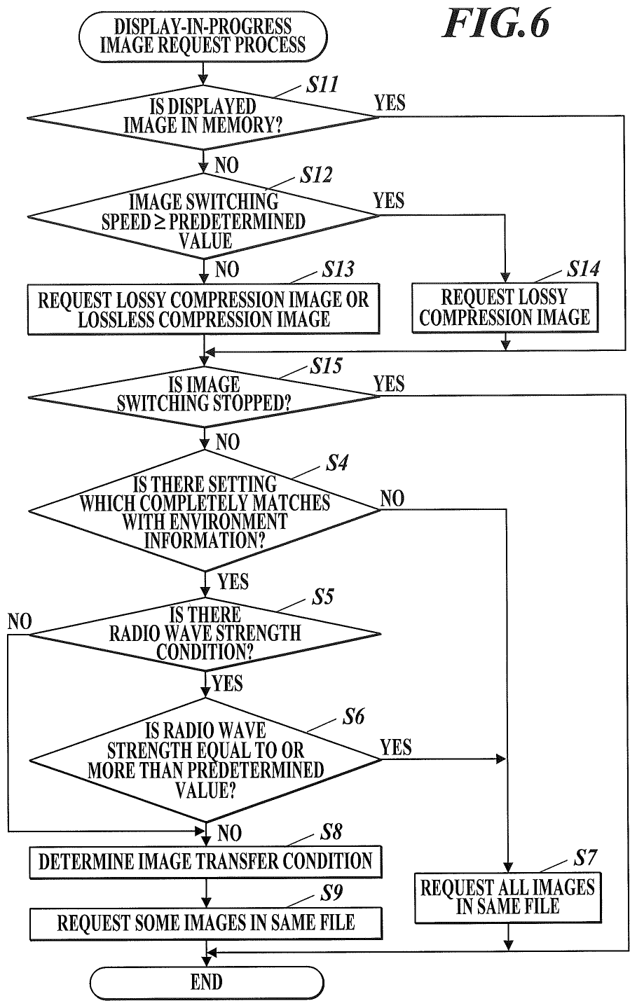

As shown in FIG. 6, in the display-in-progress image requesting process, first, it is determined whether the medical image displayed during the image switching operation is stored in the memory of the controller 21 (step S11).

In step S11, when it is determined that the displayed medical image is stored in the memory (step S11; Yes), the process jumps to the later-described step S15.

In step S11, when it is determined that the displayed medical image is not stored in the memory (step S11; No), it is determined whether the speed that the user is switching the images at present is equal to or more than the predetermined value (predetermined speed) of the switching speed described in the image transfer condition setting data (step S12).

In step S12, when it is determined that the speed that the user is switching the images is not equal to or more than the predetermined value of the switching speed described in the image transfer condition setting data (less than the predetermined value) (step S12; No), the image request signal requesting an image which is not compressed is transmitted to the server 1 through the communicator 24 (step S13), and the process advances to step S15. In step S13, the image compressed in the lossless format can be requested.

In step S12, when it is determined that the speed that the user is switching the images is equal to or more than the predetermined value of the switching speed described in the image transfer condition setting data (step S12; Yes), the image request signal requesting the lossy image is transmitted to the server 1 through the communicator 24 (step S14), and the process advances to step S15.

In step S15, it is determined whether the image switching operation is stopped.

In step S15, when it is determined that the image switching operation is stopped (step S15; Yes), the display-in-progress image request process ends.

In step S15, when it is determined that the image switching operation is not stopped (step S15; No), the processes similar to steps S4 to S9 in the above-described initial image requesting process is performed, and the display-in-progress image requesting process ends.

The client 2 configured as described above includes the following functions performed by the controller 21 executing software processes in coordination with the programs stored in the storage 13 of the server 1.

For example, the controller 21 includes the function to download through the communicator 24 the initial display image displayed immediately on the display 22 and the function to pre-download through the communicator 24 the plurality of medical images to which the display is switched to later.

According to the present embodiment, the image layout information can be downloaded together with the medical image.

The controller 21 includes the function to display the plurality of images on the display 22.

Specifically, based on the image switching operation, the display is switched from the displayed image to the medical image which is stored in the memory but is not yet displayed. Such switching of the display is repeated.

The controller 21 according to the present embodiment includes the function to divide the display 2 into a plurality of dividing regions based on the screen layout information data downloaded from the server 1, displays the plurality of medical images in one display region among the plurality of display regions, and displays the plurality of related images related to the plurality of medical images in another display region.

When the image switching operation is performed on the displayed medical image and one of the related images, the controller 21 switches the display of the one of the images and switches the other images in accordance with the above.

According to the system 100 configured as described above, when the client 2 performs the above-described initial image request process and the display-in-progress image requesting process, the server 1 transfers to the client 2 the delivered images in the amount according to the image request signal.

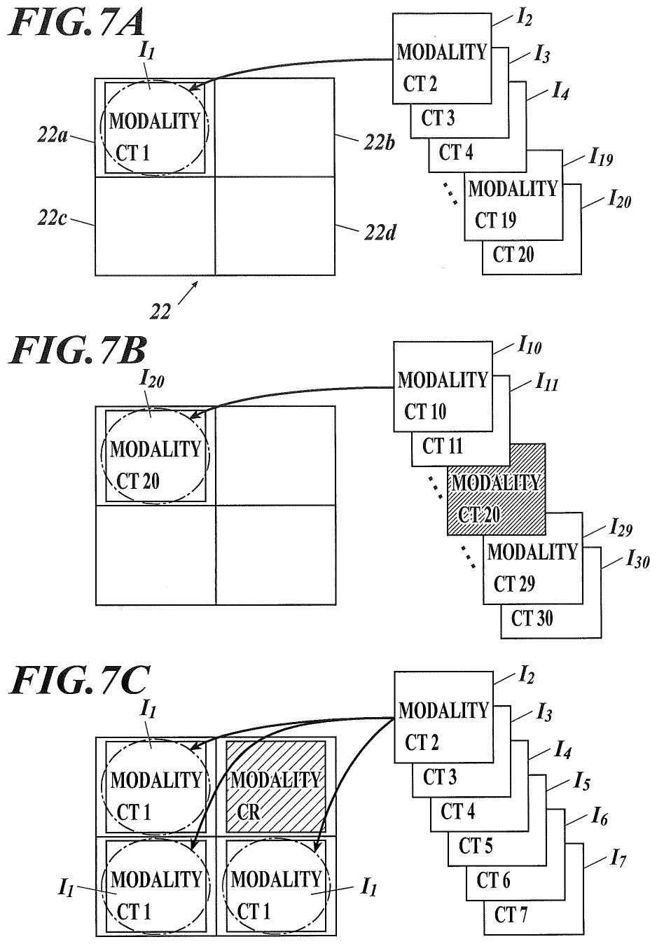

When the medical image I1 displayed on the viewer screen is the first among all images included in one file, for example, as shown in FIG. 7A, the medical images I2 to In (here n=20) for the number of pre-downloads counted from the second image are pre-downloaded. When the displayed medical image is the n-th (n≥2) as shown in FIG. 7B, the images for the number of pre-downloads counting the images prior to the displayed medical image and the images following the displayed medical image are obtained (for example, when the initial display image is the 20th image, the 10th to 19th images and the 21st to 30th images are obtained).

When there are medical images imaged by the same modality but the images are included in different files, the images are obtained from each file so that the total is to be the number of pre-downloads. (For example, as shown in FIG. 7C, when there are three image files in which the images are imaged by the same modality, and the number of pre-downloads is 20, 6 images are obtained from each file (20÷3=6.666 . . . ).)

According to the description above, according to the system 100 of the present embodiment, some of the plurality of images included in one file are transferred. Therefore, it is possible to prevent the decrease of the speed of switching the display image when the images are transferred one at a time. It is also possible to suppress the amount of downloading the wasted medical images which are not viewed.

Therefore, according to the present embodiment, in a system employing the zero footprint technique and the server rendering technique, it is possible to prevent restricted communication caused by the following, the client 2 discarding the transferred images, the large amount of medical images being wasted by the server 1 performing image processes in the middle of viewing the image, and performing unnecessary transfer of medical images.

Although embodiments of the present invention have been described and illustrated in detail, the disclosed embodiments are made for purposes of illustration and example only and not limitation. The scope of the present invention should be interpreted by terms of the appended claims.

The entire disclosure of Japanese Patent Application No. 2018-118372 filed on Jun. 22, 2018 is incorporated herein by reference in its entirety.

What is claimed is:

1. A medical image display system comprising:
a client which is able to display a plurality of images on a display; and
a server including a hardware processor which stores data of a plurality of images in a storage, which obtains environment information regarding a viewing environment of the image, which determines an image transfer condition regarding transfer of the image to the client based on the obtained environment information, which selects as target images at least some of the plurality of images stored in the storage based on the image displayed on the client and the determined image transfer condition, and which transfers the selected target images to the client using a communicator.

2. The medical image display system according to claim 1, wherein the environment information includes at least any of the following, a user name, information regarding the client, a communication speed between the communicator and the client, a connection state between the communicator and the client, status of use of a network, speed of switching the image displayed on the display, department where a user belongs, modality used to image the image, number of images included in one file, and color of image.

3. The medical image display system according to claim 1, wherein the hardware processor refers to the table showing a correspondence relation between the plurality of environment information and the plurality of image transfer conditions to determine the image transfer condition corresponding to the obtained environment information.

4. The medical image display system according to claim 1, wherein,
the plurality of images are dynamic images in which a predetermined site of a subject is imaged repeatedly to obtain a plurality of frames, and
the target images are a predetermined number of frames obtained by imaging a predetermined number of times immediately before or immediately after the frame displayed on the display.

5. The medical image display system according to claim 1, wherein,
the plurality of images are tomographic images obtained by imaging cross-sections in which a plurality of planes cut a subject, the plurality of planes positioned orthogonal to a predetermined direction and aligned in the predetermined direction with a predetermined interval in between, and
the target images are a predetermined number of tomographic images obtained by imaging a predetermined number of cross sections aligned in the predetermined direction or aligned in a direction opposite of the predetermined direction from the tomographic image displayed on the display.

6. The medical image display system according to claim 1, wherein,
the client is a zero footprint terminal, and
the hardware processor renders data of the selected target images to generate delivered images.

7. The medical image display system according to claim 1, wherein,
the client divides the display into a plurality of display regions, displays the plurality of images sequentially in one display region among the plurality of display regions, and sequentially displays in another display region a plurality of related images related to the plurality of images displayed in the one display region,
the hardware processor stores in the storage data the plurality of related images related to the plurality of images, and
the hardware processor selects at least some of the images among the plurality of images as the target image, selects the related image corresponding to each of the target images as the target related image, and transfers the images to the client.

8. The medical image display system according to claim 1, wherein,
the hardware processor compresses the image in a lossless format or a lossy format,
the hardware processor transfers the image to the client compressed in a lossy format when the client determines that a speed that the image displayed on the client is switched is a predetermined speed or more, and
the hardware processor transfers the image to the client without compression or compressed in a lossless format when the client determines that a speed that the image displayed on the client is switched is less than a predetermined speed.

9. The medical image display system according to claim 1, wherein the hardware processor transfers to the client all of the images included in one file when a predetermined condition is satisfied.
